(19) United States
(12) Patent Application Publication
(10) Pub. No.: US 2019/0142340 A1
(43) Pub. Date: May 16, 2019

(54) PHYSIOLOGICAL CONDITION MONITORING SYSTEM, DEVICE FOR COLLECTING PHYSIOLOGICAL CONDITION READINGS AND DEVICE FOR MONITORING PHYSIOLOGICAL CONDITION READINGS

(71) Applicant: Rooti Labs Limited, Cayman (KY)

(72) Inventors: MING-SHIUNG CHANG, New Taipei (TW); KO-MAI LI, New Taipei (TW)

(21) Appl. No.: 15/826,710

(22) Filed: Nov. 30, 2017

(30) Foreign Application Priority Data
Nov. 15, 2017 (CN) 201711132692.5

(51) Int. Cl. A61B 5/00; H04W 4/00; A61B 5/0408; A61B 5/0478; A61B 5/1455; A61B 5/01; A61B 5/021; A61B 5/08; A61B 5/0205

(52) U.S. Cl. CPC A61B 5/6843; H04W 4/008; A61B 5/04085; A61B 5/0478; A61B 5/14552; A61B 5/01; A61B 2560/0214; A61B 5/0816; A61B 5/002; A61B 5/681; A61B 5/746; A61B 5/0205; A61B 5/021

(57) ABSTRACT

A system for monitoring physiological condition includes a device for collecting readings and a monitoring device. The collecting device includes a number of collecting units configured to be selected human body. Based on different requirements, different numbers of collecting units can be selected to be attached to different portions of the living subject to collect readings. The selected collecting units transmit the readings to the monitoring device via wireless communication networks. The monitoring device can analyze the readings and generate a report.

### CROSS-REFERENCE TO RELATED APPLICATIONS

This application claims priority to Chinese Patent Application No. 201711132692.5 filed on Nov. 15, 2017.

### FIELD

The subject matter herein generally relates to physiological health, and particularly to a physiological condition monitoring system, a device for collecting physiological condition readings and a device for monitoring the physiological condition readings.

### BACKGROUND

Sensor devices have been used in hospital or community nursing service to detect a variety of physiological conditions of patients. Conventional sensor devices can detect some portions of living subjects via a plurality of electrodes and wires.

### BRIEF DESCRIPTION OF THE DRAWINGS

Implementations of the present technology will now be described, by way of example only, with reference to the attached figures.

FIG. 1 is a block diagram illustrating an exemplary embodiment of a system for monitoring physiological conditions, wherein the system includes a physiological data collecting device and a physiological data monitoring device.

FIG. 2 is a schematic view of an exemplary embodiment showing the physiological data collecting device and the physiological data monitoring device of FIG. 1 being used to monitor a living subject.

FIG. 3 is a block diagram of an exemplary embodiment of a data processing system.

FIG. 4 is a block diagram illustrating an exemplary embodiment of a working environment of the monitoring system.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The present disclosure, including the accompanying drawings, is illustrated by way of examples and not by way of limitation. Several definitions that apply throughout this disclosure will now be presented. It should be noted that references to “an” or “one” embodiment in this disclosure are not necessarily to the same embodiment, and such references mean “at least one.”

The term “module”, as used herein, refers to logic embodied in hardware or firmware, or to a collection of software instructions, written in a programming language, such as, Java, C, or assembly. One or more software instructions in the modules can be embedded in firmware, such as in an EPROM. The modules described herein can be implemented as either software and/or hardware modules and can be stored in any type of non-transitory computer-readable medium or other storage device. Some non-limiting examples of non-transitory computer-readable media include CDs, DVDs, BLU-RAY, flash memory, and hard disk drives. The term “comprising” means “including, but not necessarily limited to”; it specifically indicates open-ended inclusion or membership in a so-described combination, group, series, and the like.

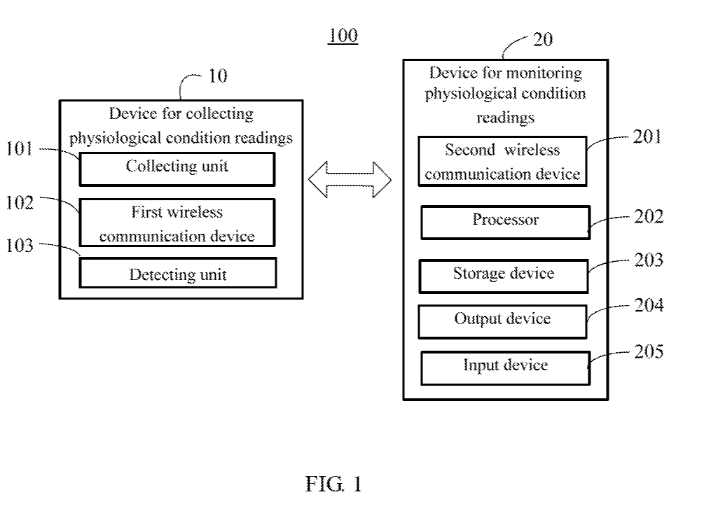

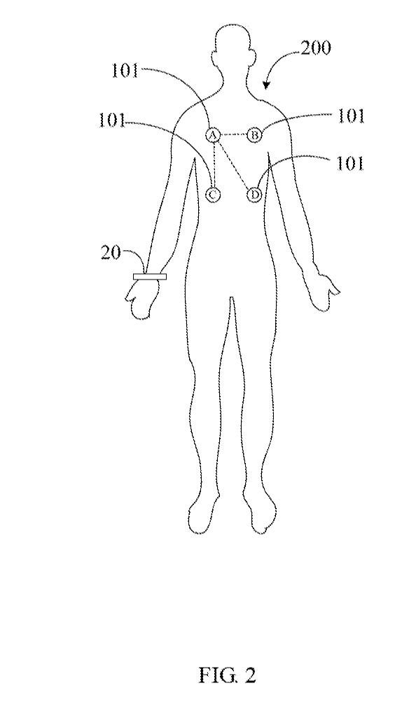

FIG. 1 illustrates a physiological condition monitoring system 100 (hereinafter “monitoring system 100”) for monitoring physiological conditions. The monitoring system 100 includes a device for collecting physiological condition readings (collecting device 10) and a device for monitoring physiological condition (monitoring device 20). The collecting device 10 includes a number of collecting units 101 configured to be selected to be attached to a portion or portions of a living subject 200 (see FIG. 2) to collect physiological condition readings of the living subject 200. Each of the collecting unit 101 communicates with the monitoring device 20 via wireless connections to transmit data to the monitoring device 20. In the embodiment, based on users' different requirements, different numbers of the collecting units 101 can be selected to be attached to different portions of the living subject 200. Taking the condition of the heart for example, each of the collecting units 101 is an electrode patch, which can be placed on the skin of a patient to collect ECG signals. The EGC signal reflects when the heart is in systolic state and diastolic state. Cardiac muscle has a spontaneous beat and rhythmic contractions. An electrical pulse sent by a cardiac conduction system can irritate the cardiac muscle fibers to make the cardiac muscle contract. Accordingly, when the cardiac conduction system generates and sends the electric pulses, the cardiac muscle produces a weak current distribution to human body. When the electrodes (collection units 11) are connected to different parts of human body, an ECG electrical pulse or signal is depicted via the electrodes (collection units 11). For example, if a first patient needs to perform a single-lead ECG two collecting units 101 can be selected to be attached to two different portions of the first patient. If a second patient needs to perform a 3-lead ECG four collecting units 101 can be selected to be attached to four different portions of the second patient, as shown in FIG. 2. If a third patient needs to perform a 12-lead ECG ten collecting units 101 can be selected to be attached to different portions of the third patient. The collecting units 101 collects electrical activity and transmits the activity to the monitoring device 20, and the monitoring device 20 analyzes the activity and generates an ECG report.

In at least one embodiment, the physiological readings can include, but are not limited to, at least one of an electrocardiogram (ECG) signal, an electroencephalogram signal, a blood pressure value, an oxygen saturation reading, a skin temperature, or data as to respiration.

In one embodiment, the monitoring device 20 can be a wristband electronic device, which can be worn on the wrist of a human body, see FIG. 2. In an alternative embodiment, the monitoring device 20 further can be, but is not limited to, other types of wearable electronic devices, for example head-mounted electronic devices, necklace-like electronic device which can be worn around the neck, or a device attached to a waistband. In other embodiments, the monitoring device 20 further can be portable electronic devices, such as mobile phone, tablet computer, or the like.

The physiological monitoring system 100 will be described in detail below.

The collecting device 10 includes a number of collecting units 101, each of the collecting unit 101 configures a first wireless communication device 102.

The collecting units 101 are configured to be selected to attached to a portion of a living subject 200 to collect physiological condition readings of the living subject 200. Based on different requirements, different numbers of the collecting units 101 can be selected to be attached to different portions of the living subject 200 to collect the physiological condition readings. In at least one embodiment, each of the collecting unit 101 is a wireless electrode patch. In a first embodiment, the physiological monitoring system 100 is an electrocardiograph, and the collecting device 10 includes a total of ten collecting units 101. Each of the collecting units 101 is an electronic patch configured to be attached to a portion of a human body to collect ECG signals. FIG. 2 shows an example of the monitoring system 100 performing a 3-lead ECG When performing the 3-lead ECG, four collecting units 101 are selected from the ten collecting units 101 to be attached to different portions of the human body as shown in FIG. 2. The four collecting units 101 are named as A, B, C, and D. In this example, the collecting unit A serves as a reference potential point, and a first lead is formed between the collecting unit A and collecting unit B. A second lead is formed between the collecting unit A and C, and a third lead is formed between the collecting unit A and D. In an alternative embodiment, each of the collecting units 101 can be a electrode patch configured to be attached to the head of the living subject 200 to collect electroencephalogram (EEG) signals of the living subject 200, and also can be an electrode patch configured to attached to the skin of the living subject 200 to collect skin temperatures, respiration data, and blood pressure values of the living subject 200. In other embodiments, each collecting unit 101 can be clamped to the finger of the living subject 200 to collect oxygen saturation data of the living subject 200.

Each first wireless communication device 102 is set in one of the collecting units 101. The first wireless communication device 102 can establish a wireless connection between the collecting unit 101 and the monitoring device 20, and transmits the physiological condition readings collected by the collecting unit 101 to the monitoring device 20. In at least one embodiment, the first wireless communication device 102 can be, but is not limited to, a BLUETOOTH module, a WI-FI module, or a ZIGBEE module.

In at least one embodiment, each collecting unit 101 has a detecting unit 103 to detect an anomaly of the collecting unit 101. The anomalous situation can be, but is not limited to, the collecting unit 101 falling from the living subject, the battery power of the collecting unit 101 is low, and the like. The detecting unit 103 further transmits the anomalous situation to the monitoring device 20 via the first wireless communication device 102. In at least one embodiment, the detecting unit 103 can include a pressure sensor to detect the collecting unit 101 falling from the living subject 200. When the collecting unit 101 is attached to the portion of the living subject 200, a pressure is generated between the collecting unit 101 and the living subject 200. If the detecting unit 103 determines that the pressure is lower than a preset pressure value, the detecting unit 103 determines that the collecting unit 101 has fallen from the living subject 200. If the detecting unit 103 so determines, the detecting unit 103 generates a prompt signal and transmits the prompt signal to the monitoring device 20 via the first wireless communication device 102. In other embodiments, the detecting unit 103 detects when the collecting unit 101 is fallen by using an infrared sensor to detect an infrared signal of the living subject. The detecting unit 103 further can detect whether the collecting unit 101 is fallen by detecting whether a physiological condition reading (e.g. the ECG signal) is detected. In the embodiment, the detecting unit 103 further can include a voltage detecting circuit to detect a voltage of a battery (not shown) of the collecting unit 101. The detecting unit 103 determines that the collecting unit 103 has low battery power if the voltage detected by the voltage detecting circuit is lower than a preset value.

The monitoring device 20 includes a second wireless communication device 201, a processor 202, a storage device 203, an output device 204, and an input device 205.

The second wireless communication device 201 can establish a wireless connection between the monitoring device 20 and the collecting units 101 of the collecting device 10, and receive the physiological condition readings from the collecting units 101. In the embodiment, the second wireless communication device 201 further receives the anomalous situation of the collecting units 101 from the collecting device 10. In the embodiment, the second wireless communication device 201 can be, but is not limited to, a BLUETOOTH module, a WI-FI module, or a ZIGBEE module.

In at least one embodiment, the processor 202 can be, but is not limited to, a central processing unit, a digital signal processor, or a single chip, for example.

In at least one embodiment, the storage device 203 can be, but is not limited to, an internal storage system, such as a flash memory, a random access memory (RAM) for temporary storage of information, and/or a read-only memory (ROM) for permanent storage of information. The storage device 203 can also be a storage system, such as a hard disk, a storage card, or a data storage medium.

The output device 204 can be, but is not limited to, a display screen, a Light Emitting Diode (LED), or a loudspeaker. The input device 205 can be, but is not limited to, a button, a touch input screen, a keyboard, or the like.

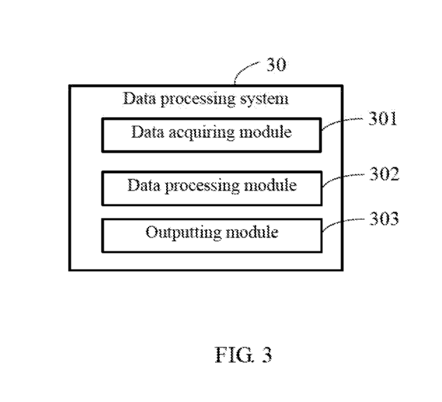

FIG. 3 illustrates an exemplary embodiment of a data processing system 30 which can be run in the monitoring device 20. In other embodiments, the data processing system 30 further can be run in a terminal device (e.g. a cloud server, a smart phone, or a tablet) which communicates with the monitoring device 20. The data processing system 30 can include a number of modules, which are collection of software instructions stored in the storage device 203 and executed by the processor 202. Referring to FIG. 3, in at least one embodiment, the data processing system 30 can include a data acquiring module 301, a data processing module 302, and an outputting module 303.

The data acquiring module 301 acquires the physiological condition readings transmitted by the collecting units 101 of the collecting device 10.

The data processing module 102 analyzes the acquired physiological condition readings and determines a quantity of the collecting units attached to the living subject 200 according to the acquired physiological condition readings, and generates a report according to the quantity of the collecting units attached to the living subject 200 and the acquired readings. In the first embodiment, the data acquiring module 101 acquires the ECG signals transmitted by the collecting units 101 attached to the living subject 200. Herein, the data processing module 12 determines a quantity of leads of the ECG according to the ECG signals and generates an ECG report according to the determined number and the ECG signals. For example, as shown in FIG. 2, the data acquiring module 101 acquires the ECG signals from the collecting units A-D, the data processing module 102 determines that there are three leads, A-B, A-C, and A-D, according to received ECG signals, and generates a three-lead ECG report according to the received ECG signals. In other embodiments, the data processing module 302 further can generate an EEG report, a skin temperature report, a report as to respiration, and/or a blood pressure report according to the acquired readings.

The outputting module 303 outputs the report via the output device 204. For example, if the output device 204 is a display screen, the outputting module 303 displays the report on the display screen. If the output device 204 is a printer, the outputting module 303 prints the report by using the printer. In at least one embodiment, the outputting module 303 further stores the report to the storage device 202.

In at least one embodiment, the data acquiring module 101 acquires the anomalous situation transmitted by the collecting device 10. The outputting module 303 outputs the anomalous situation via the output device 204, and thus warns the user about the anomaly. For example, if the anomaly is low battery, the output device 25 generates a prompt by flickering the LED, or by outputting a voice prompt using the loudspeaker.

In at least one embodiment, the input device 205 is configured for the user to input an alert when the user who wearing the monitoring system 100 feels ill. The data acquiring module 301 acquires the alert from the input device 205 and records the time that the alert is received, thus the doctor can know the time that the user feels ill.

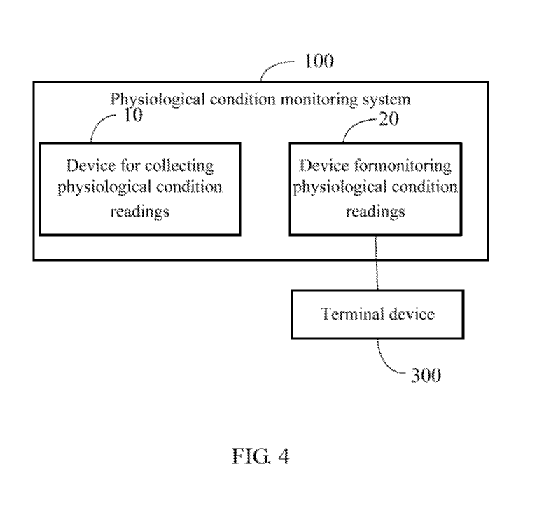

Referring to FIG. 4, in an alternative embodiment, the monitoring device 20 further can communicate with at least one terminal device 300 via the second wireless communication device 201, and can transmit the physiological parameters to the at least one terminal device 300. The at least one terminal device 300 analyzes the physiological condition readings and generates the report accordingly. In some embodiments, the monitoring device 20 transmits the report to the at least one terminal device 300. Users can obtain physiological condition readings and/or reports from the at least one terminal device 300. In at least one embodiment, the at least one terminal device 300 can be, but is not limited to, a cloud server, a web server, a personal computer (e.g. doctor's computer), or the like.

In at least one embodiment, the monitoring device 20 transmits the physiological condition readings to the at least one terminal device 300 in real time. If the connection between the monitoring device 20 and the at least one terminal device 300 is interrupted, the physiological condition readings are temporarily stored in the monitoring device 20. The temporarily stored readings are transmitted to the at least one terminal device 300 when the connection between the monitoring device 20 and the at least one terminal device 300 is restored.

In at least one embodiment, the at least one terminal device 200 further can obtain diagnosis and treatment recommendations from the network or doctors according to the readings received. The diagnosis and treatment recommendations can be transmitted to the monitoring device 20.

It is believed that the present embodiments and their advantages will be understood from the foregoing description, and it will be apparent that various changes may be made thereto without departing from the spirit and scope of the disclosure or sacrificing all of its material advantages, the examples hereinbefore described merely being exemplary embodiments of the present disclosure.

What is claimed is:

1. A device for collecting physiological condition readings comprising:
a plurality of collecting units configured to be selected to be attached to at least one portion of a living subject to collect physiological condition readings of the living subject;
each of the collecting units having a wireless communication device, the wireless communication device being configured to establish a wireless communication between the collecting unit and a device for monitoring physiological condition readings, and transmit the physiological condition readings to the device for monitoring physiological condition readings;
wherein based on different requirements, different numbers of the collecting units are selected to be attached to different portions of the living subject.

2. The device of claim 1, wherein each of the collecting unit is a wireless electrode patch.

3. The device of claim 2, wherein each collecting unit comprises a detecting unit, the detecting unit is configured to detect an anomalous situation of the collecting unit, and transmits the anomalous situation to the device for monitoring physiological condition readings via the wireless communication device.

4. The device of claim 3, wherein the anomalous situation is that the collecting unit falling from the living subject, the detecting unit is selected from a group consisting of:
a pressure sensor configured to detect whether the collecting unit has fallen from the living subject by detecting whether a pressure between the collecting unit and the living subject is less than a preset pressure value;
an infrared sensor configured to detect whether the collecting unit has fallen from the living subject by detecting whether an infrared signal of the living subject is detected; and
a detecting unit configured to detect whether the collecting unit has fallen from the living subject by detecting whether a corresponding physiological condition reading is detected.

5. The device of claim 3, wherein the anomalous situation is that a battery power of the collecting unit is low, and the detecting unit is a voltage detecting circuit.

6. The device of claim 1, wherein the physiological condition readings comprise at least one of an electrocardiogram signal, an electroencephalogram signal, a blood pressure value, an oxygen saturation reading, a skin temperature, or data as to respiration.

7. A device for monitoring physiological condition communicating with at least one device for collecting physiological condition readings, the at least one device for collecting physiological condition readings comprising a plurality of collecting units configured to be selected to attached to at least one portion of a living subject, the device for monitoring the physiological condition comprising:
a wireless communication device configured to communicate with the plurality of collecting units to receive the physiological condition readings from the collecting units;
a processor; and
a storage device storing one or more programs, when executed by the processor, the one or more programs cause the processor to:
acquire the physiological condition readings transmitted by the collecting units;
analyze the acquired physiological condition readings and determines a quantity of the collecting units attached to the living subject according to the acquired physiological condition readings, and generate a report according to the quantity of the collecting units attached to the living subject and the acquired physiological condition readings.

8. The device of claim 7, wherein the device is a wristband electronic device.

9. The device of claim 7, wherein the one or more programs further cause the processor to: acquire an anomalous situation of the collecting units from the device for collecting physiological condition readings, and output the anomalous situation via an output device.

10. The device of claim 7, further comprising an input device configured for a user to input an alert when the user who wearing the device for monitoring the physiological condition readings feels ill, wherein the one or more programs further cause the processor to: acquire the alert from the input device and record a time that the alert is received.

11. The device of claim 7, further communicating with at least one terminal device via the wireless communication device, and transmitting the physiological condition readings to the at least one terminal device to generate the report by the at least one terminal device.

12. The device of claim 11, further transmitting the physiological condition readings to the at least one terminal device in real time; if a connection between the device for monitoring the physiological condition readings and the at least one terminal device is interrupted, the physiological condition readings are temporarily stored in the device for monitoring the physiological condition readings, and the temporarily stored physiological condition readings are transmitted to the at least one terminal device when the connection is restored.

13. A physiological condition monitoring system comprising:
a device for collecting physiological condition readings comprising a plurality of collecting units configured to be selected to be attached to at least one portion of a living subject to collect physiological condition readings of the living subject; each of the collecting units having a first wireless communication device, the first wireless communication device being configured to establish a wireless communication between the collecting unit and a device for monitoring physiological condition readings, and transmit the physiological condition readings to the device for monitoring physiological condition readings; wherein based on different requirements, different numbers of the collecting units are selected to be attached to different portions of the living subject; and
a device for monitoring physiological condition readings comprising:
a second wireless communication device configured to communicate with the first wireless communication device and receives the physiological condition readings transmitted by the collecting units;
a processor; and
a storage device storing one or more programs, when executed by the processor, the one or more programs cause the processor to:
acquire the physiological condition readings transmitted by the collecting units; and
analyze the acquired physiological condition readings and determines a quantity of the collecting units attached to the living subject according to the acquired physiological condition readings, and generate a report according to the quantity of the collecting units attached to the living subject and the acquired physiological condition readings.

14. The physiological condition monitoring system of claim 13, wherein each collecting unit comprises a detecting unit, the detecting unit is configured to detect an anomalous situation of the collecting unit, and transmits the anomalous situation to the device for monitoring physiological condition readings via the wireless communication device.

15. The physiological condition monitoring system of claim 13, the physiological condition readings comprise at least one of an electrocardiogram signal, an electroencephalogram signal, a blood pressure value, an oxygen saturation reading, a skin temperature, or data as to respiration.

16. The physiological condition monitoring system of claim 13, wherein the device for monitoring the physiological condition readings is a wristband electronic device.

17. The physiological condition monitoring system of claim 13, wherein the device for monitoring the physiological condition readings further comprises an input device configured for a user to input an alert when the user who wearing the device for monitoring the physiological condition readings feels ill, wherein the one or more programs further cause the processor to: acquire the alert from the input device and record a time that the alert is received.

18. The physiological condition monitoring system of claim 13, wherein the device for monitoring the physiological condition readings further communicates with at least one terminal device via the second wireless communication device, and transmits the physiological condition readings to the at least one terminal device to generate the report by the at least one terminal device.

19. The physiological condition monitoring system of claim 18, wherein the device for monitoring the physiological condition readings transmits the physiological condition readings to the at least one terminal device in real time; if a connection between the the device for monitoring the physiological condition readings and the at least one terminal device is interrupted, the physiological condition readings are temporarily stored in the device for monitoring the physiological condition readings, and the temporarily stored physiological condition readings are transmitted to the at least one terminal device when the connection is restored.
